(19) United States
(12) Patent Application Publication
(10) Pub. No.: US 2005/0053953 A1
(43) Pub. Date: Mar. 10, 2005

(54) Novel human genes and proteins encoded thereby

(76) Inventors: Alex Turner, The Woodlands, TX (US); Brian Zambrowicz, The Woodlands, TX (US); Michael Nehls, Stockdorf (DE); Glenn A. Friedrich, The Woodlands, TX (US); Arthur T. Sands, The Woodlands, TX (US)

Correspondence Address: Lance K. Ishimoto;LEXICON GENETICS INCORPORATED, 8800 Technology Forest Place, The Woodlands, TX, 77381, US

(21) Appl. No.: 10/681,744

(22) Filed: Oct. 8, 2003

Related U.S. Application Data
(63) Continuation of application No. 09/602,833, filed on Jun. 23, 2000, now abandoned.
(60) Provisional application No. 60/140,627, filed on Jun. 23, 1999.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention relates to novel human genes involved in the regulation of signal transduction pathways, and the proteins and polypeptides encoded thereby. In particular, the invention relates to polynucleotides that encode the polypeptides, the polypeptides, antibodies directed to the polypeptides, and methods of diagnosis and treatment of various disorders, including disorders involving the inappropriate regulation of a signal transduction mechanism, for example cancer.

This application claims priority under 35 U.S.C. §119(e) to U.S. Provisional Application No. 60/140,627, filed Jun. 23, 1999, which is hereby incorporated by reference in its entirety.

### 1. INTRODUCTION

The present invention relates to the discovery, identification and characterization of novel human polynucleotides (hereinafter referred to collectively as “SGT4” polynucleotides or genes) that encode proteins, and, more particularly, proteins involved in signal transduction mechanisms. The invention encompasses the described polynucleotides and novel related polynucleotides, vectors and compositions comprising the polynucleotides, host cell expression systems, the products encoded by the polynucleotides and genes (hereinafter referred to collectively as “SGT4” proteins, peptides and polypeptides), modified and fusion proteins, variants and homologs of the encoded proteins, antibodies to the encoded proteins and peptides, and genetically engineered animals that lack the disclosed genes, or overexpress the disclosed genes, compounds that bind to SGT4 or a cognate ligand, binding partner or substrate of SGT4 (e.g., antagonists and agonists of the proteins), and other compounds that modulate the expression, processing or activity of the proteins encoded by the disclosed genes that can be used for diagnosis, drug screening, clinical trial monitoring, the treatment of physiological or behavioral disorders or dysfunctions, cancer, and infectious disease.

### 2. BACKGROUND OF THE INVENTION

Proteins are integral components of the various systems used by the body to effect, monitor and regulate different bodily functions. An increasingly large number of proteins involved in signal transduction mechanisms have been identified in recent years, and these proteins have been shown to control different steps of pathways regulating cell survival, proliferation and differentiation. In many cases, the mutation or inappropriate expression of such a protein can result in cancer. It follows that these proteins constitute attractive targets for the development of therapeutic agents, particularly anti-cancer drugs. Such therapeutic agents can take the form of molecules that interact with such a protein or its ligands, or otherwise regulate, attenuate or enhance the expression or activity of such a protein. Alternatively, the proteins themselves, or nucleic acids encoding such proteins, can be used as therapeutic agents. Furthermore, the detection of a mutation or altered expression levels of such a protein can serve as a marker indicating the existence of a disorder in a subject, e.g., cancer, or indicating a subject's propensity for such a disorder.

One class of proteins that has been shown to play an important role in signal transduction pathways and development consists of the leucine rich repeat domain (LLRa) containing proteins. Leucine-rich repeats are short sequence motifs which mediate protein-protein interactions. Kobe and Deisenhofer, Opin. Struct. Biol. (1995) 5(3):409-16. Examples of LLRa containing proteins include Ras suppressor protein-1 (RSU-1), which is thought to play a role in the Ras signal transduction pathway (Cutler et al., Mol. Cell. Biol. (1992) 12(9):3570-76) and the flightless-1 protein homolog, thought to play a role in embryonic cellularization by interacting with both the cytoskeleton and other cellular components (Campbell et al., Proc. Natl. Acad. Sci. USA (1993) 90:11386-11390). The identification of novel (LLRa) containing proteins and the genes encoding them is of inherent value to the biomedical research community, since these novel proteins and genes can serve as the basis for the development of novel therapeutic agents, particularly for the treatment of diseases involving aberrant or improperly regulated signal transduction mechanisms, e.g., cancer.

### 3. SUMMARY OF THE INVENTION

The present invention relates to the discovery, identification and characterization of novel human polynucleotides (hereinafter referred to collectively as “SGT4” polynucleotides or genes) that encode proteins, and, more particularly, proteins involved in signal transduction mechanisms. The invention is based, in part, on Applicants' discovery that the encoded proteins (hereinafter referred to collectively as “SGT4” proteins, peptides or polypeptides) share substantial sequence homology with the leucine rich repeat domain (LLRa) containing proteins, particularly RSU-1 and the flightless-I protein homolog. While SGT4 shares sequence homology with other LLRa-containing proteins, its primary sequence is unique. Its expression is detected in various human tissues, and at particularly high levels in skeletal muscle and heart.

The invention encompasses the polynucleotides presented in the Sequence Listing and Figures, and: (a) polynucleotides that encode mammalian homologs of the described genes, including the specifically described SGT4 variants, and their gene products; (b) polynucleotides that encode one or more portions of SGT4 that correspond to functional domains, and the polypeptide products specified by such nucleotide sequences, including but not limited to the novel regions of any active domain(s); (c) isolated polynucleotides that encode mutant versions, engineered or naturally occurring, of the described polypeptides in which all or a part of at least one domain is deleted or altered, and the polypeptide products specified by such nucleotide sequences, including but not limited to soluble proteins and peptides in which all or a portion of the signal sequence in deleted; and (d) polynucleotides that encode chimeric fusion proteins containing all or a portion of a coding region of an SGT4, or one of its domains (e.g., a receptor binding domain, accessory protein/self-association domain, etc.) fused to another peptide or polypeptide.

The invention also encompasses vectors, compositions and host cell expression systems comprising the above-mentioned polynucleotides, the products encoded by the polynucleotides and genes (hereinafter referred to collectively as “SGT4” proteins, polypeptides and polypeptides), including proteins having the amino acid sequences presented in the Sequence Listing and Figures, modified and fusion proteins, variants and homologs of the encoded proteins, antibodies to the encoded proteins and peptides, and genetically engineered animals that lack the disclosed genes, or overexpress the disclosed genes, compounds that bind to SGT4 or a cognate ligand, binding partner or substrate of SGT4 (e.g., antagonists and agonists of the proteins), and other compounds that modulate the expression, processing or activity of the proteins encoded by the disclosed genes that can be used for diagnosis, drug screening, clinical trial monitoring, the treatment of physiological or behavioral disorders or dysfunctions, cancer, and infectious disease.

The invention also encompasses nucleotide sequences that can be used to inhibit the expression of SGT4 (e.g., antisense and ribozyme molecules, and gene or regulatory sequence replacement constructs) or to enhance the expression of the described SGT4-encoding genes, i.e., SGT4 genes (e.g., expression constructs that place the described gene under the control of a strong promoter system), and transgenic animals that express an SGT4 transgene. Additionally, “knock-out” animals are contemplated (which can be conditional) that have been engineered such that they do not express a functional SGT4 gene (see, for example, PCT Applic. No. PCT/US98/03243, filed Feb. 20, 1998, herein incorporated by reference). Another aspect of the present invention includes cells and animals that have specifically engineered mutations (point mutations, overexpression of an SGT4 gene, etc.) in the genes encoding the presently described proteins and polypeptides.

Further, the present invention also relates to methods of using the described polypeptides and their coding sequences for the identification of compounds that modulate, i.e., act as agonists or antagonists, of SGT4 expression or SGT4 activity, or that interfere with or affect the interaction of SGT4 with a cognate ligand, binding partner or substrate. Such compounds can be used as therapeutic agents for the treatment of any of a wide variety of symptomatic representations of biological disorders or imbalances.

The invention further encompasses methods for producing and using the disclosed polynucleotides and polypeptides in a variety of research, diagnostic and therapeutic applications, methods for identifying compounds and factors that modulate the expression, processing or activity of the disclosed polynucleotides and polypeptides, and methods for detecting and quantitating levels of the disclosed polynucleotides and polypeptides, as well as a variety of other uses that flow naturally from the instant disclosure and which would be readily apparent to one of skill the art.

### 4. BRIEF DESCRIPTION OF THE FIGURES

FIG. 1. The cDNA sequence of SGT4-1 (SEQ ID NO: 1).

FIG. 2. The amino acid sequence of SGT4-1 (SEQ ID NO: 2), which is the polypeptide predicted by the open reading frame of SGT4-1.

FIG. 3. The cDNA sequence of SGT4-2 (SEQ ID NO: 3).

FIG. 4. The amino acid sequence of SGT4-2 (SEQ ID NO: 4), which is the polypeptide predicted by the open reading frame of SGT4-2.

### 5. DETAILED DESCRIPTION OF THE INVENTION

5.1. Nucleotide Sequences Encoding SGT4

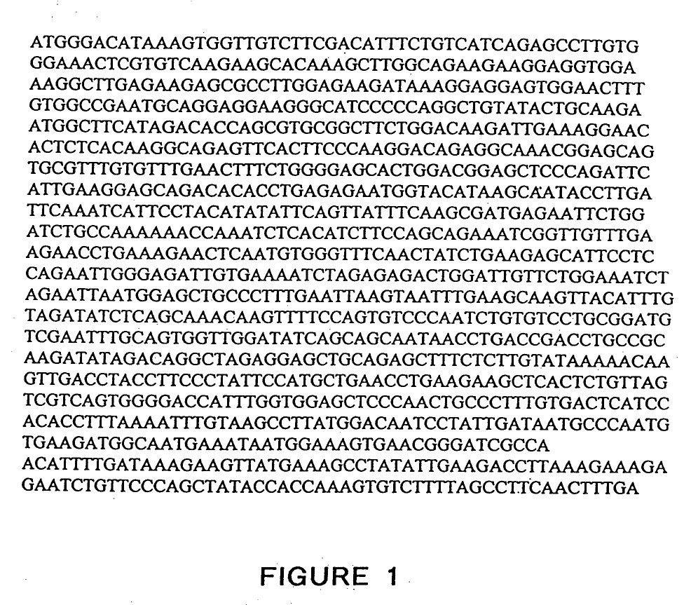

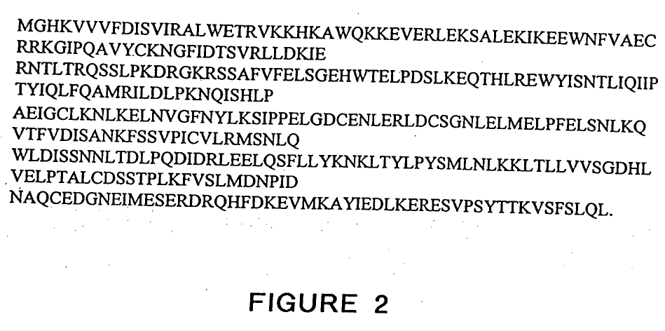

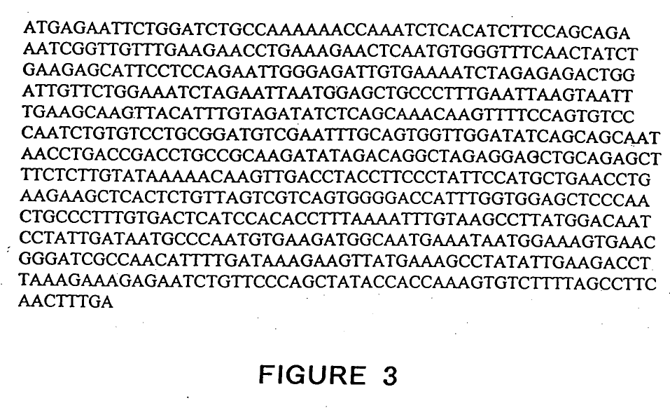

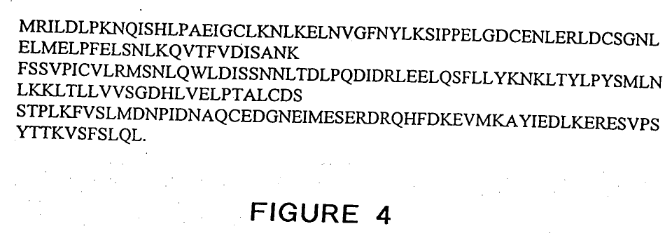

The present invention relates to nucleic acid molecules that encode polypeptides referred to collectively as “SGT4.” In a specific embodiment, cDNA sequences encoding 2 variants of SGT4 (SGT4-1 and SGT4-2) were determined, and their nucleotide and deduced amino acid sequences characterized. cDNA sequences encoding the SGT4 variants SGT4-1 and SGT4-2 are provided in FIGS. 1 and 3 (SEQ ID NOS: 1 and 3), and the corresponding deduced amino acid sequences are provided in FIGS. 2 and 4 (SEQ ID NOS: 2 and 4). The described polynucleotide sequences were obtained in part from human gene trap libraries generated essentially as described in U.S. Patent Application Ser. Nos. 60/095,989 and 09/276,533, both incorporated herein by reference, and in part by screening human cDNA libraries. Alternatively, the polynucleotides of the invention can be obtained using standard techniques well known to those skilled in the art such as, for example, hybridization screening and PCR methodology. Preferred sources of expressed SGT4 encoding polynucleotides include skeletal muscle and heart.

SGT4 shares substantial sequence homology with other leucine rich repeat domain (LLRa) containing proteins, particularly RSU-1 (Cutler et al., Mol. Cell. Biol. (1992) 12(9):3570-76) and the flightless-I protein homolog (Campbell et al., Proc. Natl. Acad. Sci. USA (1993) 90:11386-11390). Nevertheless, the nucleotide coding sequences and deduced amino acid sequences of SGT4 are structurally unique. In accordance with the invention, any nucleotide sequence which encodes the amino acid sequence of the human SGT4 gene products can be used to generate recombinant molecules which direct the expression of SGT4. Additionally, the invention also relates to a fusion polynucleotide between an SGT4 coding sequence and a second coding sequence for a heterologous protein.

In order to clone full length homologous cDNA sequences from any species encoding the entire SGT4 cDNA or to clone family members or variant forms such as allelic variants, labeled DNA probes made from fragments corresponding to any part of the cDNA sequences disclosed herein may be used to screen a cDNA library derived from a cell or tissue type believed to express SGT4, e.g., skeletal muscle or heart tissue. More specifically, oligonucleotides corresponding to either the 5′ or 3′ terminus of the coding sequence may be used to obtain longer nucleotide sequences. Briefly, the library may be plated out to yield a maximum of 30,000 pfu for each 150 mm plate. Approximately 40 plates may be screened. The plates are incubated at 37° C. until the plaques reach a diameter of 0.25 mm or are just beginning to make contact with one another (3-8 hours). Nylon filters are placed onto the soft top agarose and after 60 seconds, the filters are peeled off and floated on a DNA denaturing solution consisting of 0.4N sodium hydroxide. The filters are then immersed in neutralizing solution consisting of 1M Tris HCl, pH 7.5, before being allowed to air dry. The filters are prehybridized in casein hybridization buffer containing 10% dextran sulfate, 0.5M NaCl, 50 mM Tris HCL, pH 7.5, 0.1% sodium pyrophosphate, 1% casein, 1% SDS, and denatured salmon sperm DNA at 0.5 mg/ml for 6 hours at 60° C. The radiolabelled probe is then denatured by heating to 95° C. for 2 minutes and then added to the prehybridization solution containing the filters. The filters are hybridized at 60° C. for 16 hours. The filters are then washed in 1× wash mix (10× wash mix contains 3M NaCl, 0.6M Tris base, and 0.02M EDTA) twice for 5 minutes each at room temperature, then in 1× wash mix containing 1% SDS at 60° C. for 30 minutes, and finally in 0.3× wash mix containing 0.1% SDS at 60° C. for 30 minutes. The filters are then air dried and exposed to x-ray film for autoradiography. After developing, the film is aligned with the filters to select a positive plaque. If a single, isolated positive plaque cannot be obtained, the agar plug containing the plaques will be removed and placed in lambda dilution buffer containing 0.1M NaCl, 0.01M magnesium sulfate, 0.035M Tris HCl, pH 7.5, 0.01% gelatin. The phage may then be replated and rescreened to obtain single, well isolated positive plaques. Positive plaques may be isolated and the cDNA clones sequenced using primers based on the known cDNA sequence. This step may be repeated until a full length cDNA is obtained.

It may be necessary to screen multiple cDNA libraries from different tissues to obtain a full length cDNA. In the event that it is difficult to identify cDNA clones encoding the complete 5′ terminal coding region, an often encountered situation in cDNA cloning, the RACE (Rapid Amplification of cDNA Ends) technique may be used. RACE is a proven PCR-based strategy for amplifying the 5′ end of incomplete cDNAs. 5′-RACE-Ready RNA synthesized from human placenta containing a unique anchor sequence is commercially available (Clontech). To obtain the 5′ end of the cDNA, PCR is carried out on 5′-RACE-Ready cDNA using the provided anchor primer and the 3′ primer. A secondary PCR is then carried out using the anchored primer and a nested 3′ primer according to the manufacturer's instructions. Once obtained, the full length cDNA sequence may be translated into amino acid sequence and examined for certain landmarks such as a continuous open reading frame flanked by translation initiation and termination sites, a potential signal sequence and finally overall structural similarity to the SGT4 genes disclosed herein.

Alternatively, a labeled probe may be used to screen a genomic library derived from any organism of interest using appropriate stringent conditions as described infra.

Isolation of an SGT4 coding sequence or a homologous sequence may be carried out by the polymerase chain reactions (PCR) using two degenerate oligonucleotide primer pools designed on the basis of the SGT4 coding sequences disclosed herein. The template for the reaction may be cDNA obtained by reverse transcription (RT) of mRNA prepared from, for example, human or non-human cell lines or tissues known or suspected to express an SGT4 gene allele.

The PCR product may be subcloned and sequenced to ensure that the amplified sequences represent the sequences of an SGT4 coding sequence. The PCR fragment may then be used to isolate a full length cDNA clone by a variety of methods. For example, the amplified fragment may be labeled and used to screen a bacteriophage cDNA library. Alternatively, the labeled fragment may be used to isolate genomic clones via the screening of a genomic library.

PCR technology may also be utilized to isolate full length cDNA sequences. For example, RNA may be isolated, following standard procedures, from an appropriate cellular or tissue source. A RT reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated.

A cDNA clone of a mutant or allelic variant of the SGT4 gene may be isolated, for example, by using PCR. In this case, the first cDNA strand may be synthesized by hybridizing an oligo-dT oligonucleotide to mRNA isolated from tissue known or suspected to be expressed in an individual putatively carrying the mutant SGT4 allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant SGT4 allele to that of the normal SGT4 allele, the mutation(s) responsible for the loss or alteration of function of the mutant SGT4 gene product can be ascertained.

Alternatively, a genomic library can be constructed using DNA obtained from an individual suspected of or known to carry a mutant SGT4 allele, or a cDNA library can be constructed using RNA from a tissue known, or suspected, to express a mutant SGT4 allele. An unimpaired SGT4 gene or any suitable fragment thereof may then be labeled and used as a probe to identify the corresponding mutant SGT4 allele in such libraries. Clones containing the mutant SGT4 gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant SGT4 allele in an individual suspected of or known to carry such a mutant allele. In this manner, gene products made by the putatively mutant tissue may be expressed and screened using standard antibody screening techniques in conjunction with antibodies raised against the normal SGT4 gene product, as described, below, in Section 5.5. (For screening techniques, see, for example, Harlow and Lane, eds., 1988, “Antibodies: A Laboratory Manual”, Cold Spring Harbor Press, Cold Spring Harbor.)

In cases where an SGT4 mutation results in an expressed gene product with altered function (e.g., as a result of a missense), a polyclonal set of anti-SGT4 gene product antibodies are likely to cross-react with the mutant SGT4 gene product. Library clones detected via their reaction with such labeled antibodies can be purified and subjected to sequence analysis according to methods well known to those of skill in the art.

As used herein, the terms nucleic acid, polynucleotide and nucleotide are interchangeable and refer to any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

The terms nucleic acid, polynucleotide and nucleotide also specifically includes nucleic acids composed of bases other than the five biologically occurring bases (adenine, guanine, thymine, cytosine and uracil). For example, a polynucleotide of the invention might contain at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Furthermore, a polynucleotide of the invention may comprise at least one modified sugar moiety selected from the group including but not limited to arabinose, 2-fluoroarabinose, xylulose, and hexose.

It is not intended that the present invention be limited by the source of the polynucleotide. The polynucleotide can be from a human or non-human mammal, derived from any recombinant source, synthesized in vitro or by chemical synthesis. The nucleotide may be DNA or RNA and may exist in a double-stranded, single-stranded or partially double-stranded form.

Nucleic acids useful in the present invention include, by way of example and not limitation, oligonucleotides such as antisense DNAs and/or RNAs; ribozymes; DNA for gene therapy; DNA and/or RNA chimeras; various structural forms of DNA including single-stranded DNA, double-stranded DNA, supercoiled DNA and/or triple-helix DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, Oligonucleotide Synthesis: A Practical Approach, IRL Press, Oxford, England). RNAs may be produce in high yield via in vitro transcription using plasmids such as SP65 (Promega Corporation, Madison, Wis.).

The present invention includes any mRNA transcript encoded by the SGT4 genes of the invention, including in particular, mRNA transcripts resulting from alternative splicing or processing of mRNA precursors. Northern analysis of various tissue types, particularly skeletal muscle and heart, has revealed the existence of mRNA transcripts containing a SGT4 encoding nucleotide sequence of the present invention, or a fragment thereof, of various sizes. In particular, such mRNA transcripts of about 5 kb, 1.7 kb and 2.3 kb have been identified.

In some circumstances, as where increased nuclease stability is desired, nucleic acids having modified internucleoside linkages may be preferred. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), dimethylene-sulfoxide (—CH₂—SO—CH₂), dimethylene-sulfone (—CH₂—SO₂—CH₂), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein).

In some embodiments of the present invention, the nucleotide is an α-anomeric nucleotide. An α-anomeric nucleotide forms specific double-stranded hybrids with complementary RNA in which, contrary to the usual β-units, the strands run parallel to each other (Gautier et al., 1987, Nucl. Acids Res. 15:6625-6641). The nucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

The nucleic acids may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the DNA to be purified.

The nucleic acid itself may act as a therapeutic agent, such as for example an antisense DNA that inhibits mRNA translation, or the nucleic acid may encode an SGT4 capable of inducing a therapeutic affect upon expression in a subject. These gene products can potentially function as therapeutic molecules in a variety of contexts, for example, as cytokines, chemokines, signaling molecules, membrane proteins, transcription factors, intracellular proteins, cytokine binding proteins, and the like.

The invention also relates to isolated or purified polynucleotides having at least 12 nucleotides (i.e., a hybridizable portion) of an SGT4 coding sequence or its complement. In other embodiments, the polynucleotides contain at least 25 (continuous) nucleotides, 50 nucleotides, 100 nucleotides, 150 nucleotides, or 200 nucleotides of an SGT4 coding sequence, or a full-length SGT4 coding sequence. Nucleic acids can be single or double stranded. Additionally, the invention relates to polynucleotides that selectively hybridize to a complement of the foregoing coding sequences. In preferred embodiments, the polynucleotides contain at least 12, 25, 50, 100, 150 or 200 nucleotides or the entire length of an SGT4 coding sequence.

In a specific embodiment, a polynucleotide which hybridizes to an SGT4 coding sequence (e.g., a polynucleotide having the sequence of SEQ ID NOS: 1 or 3) or its complement under conditions of low stringency is provided. By way of example and not limitation, exemplary conditions of low stringency are as follows (Shilo and Weinberg, 1981, Proc. Natl. Acad. Sci. USA 78:6789-6792): Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

In another specific embodiment, a polynucleotide which hybridizes to an SGT4 coding sequence or its complement under conditions of high stringency is provided. By way of example and not limitation, exemplary conditions of high stringency are as follows: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency which may be used are well known in the art.

In another specific embodiment, a polynucleotide which hybridizes to an SGT4 coding sequence or its complement under conditions of moderate stringency is provided. Exemplary conditions of moderate stringency are as follows: Filters containing DNA are pretreated for 6 h at 55° C. in a solution containing 6×SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 h at 55° C., and then washed twice for 30 minutes at 60° C. in a solution containing 1×SSC and 0.1% SDS. Filters are blotted dry and exposed for autoradiography. Other conditions of moderate stringency which may be used are well-known in the art.

The invention also encompasses nucleotide sequences that encode a mutant of SGT4, peptide fragments of SGT4, truncated forms of SGT4, and SGT4 fusion proteins. These include, but are not limited to nucleotide sequences encoding the mutant proteins and polypeptides described in Section 5.2; polypeptides or peptides corresponding to one or more domains of SGT4, or portions of these domains; truncated forms of SGT4, in which one or more of the domains is deleted; or a truncated, nonfunctional SGT4. Nucleotides encoding fusion proteins may include, but are not limited to, full length SGT4 sequences, truncated forms of SGT4, or nucleotides encoding peptide fragments of SGT4 fused to an unrelated protein or peptide, such as for example, a SGT4 domain fused to an Ig Fc domain which increases the stability and half life of the resulting fusion protein (e.g., SGT4-Ig) in the bloodstream; or an enzyme such as a fluorescent protein or a luminescent protein which can be used as a marker.

The invention further encompasses polynucleotides encoding soluble derivatives of membrane-associated forms of SGT4. Such soluble derivatives can be engineered by excising the membrane-anchoring region of the protein, e.g., creating a derivative of SGT4 lacking a transmembrane domain, comprising only an extracellular and/or intracellular domain.

Furthermore, the instant invention encompasses SGT4 polynucleotide variants that have been generated, at least in part, by some form of directed evolution, e.g., gene shuffling and/or recursive sequence recombination, described in U.S. Pat. Nos. 5,605,793 and 5,837,458, incorporated by reference herein in their entirety. For example, using such techniques one can use an SGT4 encoding sequence, or a plurality of SGT4 encoding sequences, as the starting point for the generation of novel sequences encoding functionally and/or structurally similar proteins with altered functional and/or structural characteristics.

The invention encompasses highly related gene homologs of the SGT4 encoding polynucleotide sequences described above. Highly related gene homologs are polynucleotides encoding proteins that are at least 30% identical, or at least 40% identical, preferably 50% identical, more preferably 60% identical, even more preferably 70% or even 80% identical, and most preferably 90% identical, at the amino acid level to the disclosed SGT4 proteins. Percent similarity may be determined, for example, by comparing sequence information using the BLAST computer program, version 2.0, available on the World-Wide Web at http://www.ncbi.nlm.nih.gov. For a description of BLAST, see Altschul et al., J. Mol. Biol. 215:403-10 (1990); Altschul et al., Nucleic Acids Res. 25:3389-3402 (1997). Typical parameters for determining the similarity of two sequences using BLAST 2.0 are a reward for match of 1, penalty for mismatch of 2, open gap and extension gap penalties of 5 and 2, respectively, a gap dropoff of 50, and a word size of 11. Highly related homologs can encode proteins sharing functional activities with SGT4. Other gene homologs are those genes that encode proteins having 100% identity with SGT4 over 6 consecutive amino acids, and more preferably 8 amino acids, yet more preferably 15 amino acids, or even 20 amino acids. Alternatively, percent homology may be determined using the GAP computer program, version 6.0 described by Devereux et al., Nucl. Acids. Res., 12:387 (1984). The GAP program utilizes the alignment method of Neeldeman and Wunsch, J. Mol. Biol. 48:443 (1970), as revised by Smith and Waterman, Adv. Appl. Math, 2:482 (1970). Percent similarity may be determined, for example, by comparing sequence information using the BLAST computer program, version 2.0, available on the World-Wide Web at http://www.ncbi.nlm.nih.gov.

The invention also encompasses (a) DNA vectors that contain any of the foregoing SGT4 coding sequences and/or their complements (i.e., antisense); (b) DNA expression vectors that contain any of the foregoing SGT4 coding sequences operatively associated with a regulatory element that directs the expression of the coding sequences; (c) genetically engineered host cells that contain any of the foregoing SGT4 coding sequences operatively associated with a regulatory element that directs the expression of the coding sequences in the host cell; and (d) genetically engineered host cells that express an endogenous SGT4 gene under the control of an exogenously introduced regulatory element (i.e., gene activation).

The present invention also encompasses polynucleotide sequences encoding SGT4 variants that are the product of alternatively spliced SGT4 mRNA transcripts. SGT4 mRNA transcripts of approximately 7.5 kb, 1.7 kb and 2.3 kb have been identified by Northern analysis in various tissue types, including skeletal muscle and heart.

5.2. Products Encoded by the Polynucleotides Disclosed Herein

In accordance with the invention, an SGT4 polynucleotide which encodes full length SGT4 polypeptides, mutant polypeptides, peptide fragments of SGT4, SGT4 fusion proteins or functional equivalents thereof, may be used to generate recombinant DNA molecules that direct the expression of SGT4 polypeptides, mutant polypeptides, SGT4 peptide fragments, SGT4 fusion proteins or a functional equivalent thereof, in appropriate host cells. Such polynucleotides, as well as other polynucleotides which selectively hybridize to at least a part of such SGT4 polynucleotides or their complements, may also be used to produce SGT4 polypeptides or they may be used in nucleic acid hybridization assays, such as Southern and Northern blot analyses, etc. The polypeptide products encoded by such polynucleotides may be naturally occurring or altered by molecular manipulation of the coding sequence.

Due to the inherent degeneracy of the genetic code, other DNA sequences which encode substantially the same or a functionally equivalent SGT4 amino acid sequence (e.g., having the sequence of SEQ ID NOS: 2 or 4) may be used in the practice of the invention for the cloning and expression of SGT4 proteins. Such DNA sequences include those which are capable of hybridizing to the human or mouse SGT4 coding sequence or its complementary sequence under low, moderate or high stringency conditions as described in Section 5.1.

The invention also encompasses proteins that are functionally equivalent to the SGT4 proteins encoded by the nucleotide sequences described in Section 5.1, as judged by any of a number of criteria, including, but not limited to, the ability to bind a receptor, ligand, binding partner, or substrate of SGT4, the ability to affect an identical or complementary signal transduction pathway, the ability to induce a therapeutic effect, the ability to effect a change in cellular metabolism (e.g., ion flux, tyrosine phosphorylation, etc.), or change in phenotype when the SGT4 equivalent is similarly expressed or mutated in an appropriate cell type (such as the amelioration, prevention or delay of a biochemical, biophysical, or overt phenotype). Such functionally equivalent SGT4 proteins include, but are not limited to, SGT4 proteins including additions, deletions or substitutions of amino acid residues within the amino acid sequence encoded by the SGT4 nucleotide sequences described above in Section 5.1, where the change does not affect the function of the protein, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

The nucleotide sequences of the invention may be engineered in order to alter an SGT4 coding sequence for a variety of ends, including but not limited to, alterations which modify processing and expression of the gene product. For example, mutations may be intro-duced using techniques which are well known in the art, e.g., site-directed mutagenesis, to insert new restriction sites, to alter glycosylation patterns, phosphorylation, etc. Alterations may also affect one or more biologic activities of SGT4. For example, cysteine residues can be deleted or substituted with another amino acid to eliminate disulfide bridges.

Based on the domain organization of the SGT4 protein, a large number of SGT4 mutant polypeptides can be constructed by rearranging the nucleotide sequences that encode the SGT4 domains.

In another embodiment of the invention, an SGT4 coding sequence, a modified SGT4 coding sequence or a truncated SGT4 coding sequence corresponding to a specific domain may be ligated to a heterologous sequence to produce a fusion protein. For example, for screening of peptide libraries for molecules that bind SGT4, it may be useful to encode a chimeric SGT4 protein expressing a heterologous epitope that is recognized by a commercially available antibody. A fusion protein may also be engineered to contain a cleavage site located between an SGT4 sequence and the heterologous protein sequence, so that the SGT4 may be cleaved and separated from the heterologous moiety. A heterologous moiety includes, but is not limited to, immunoglobulin constant domain which prolongs in vivo half-life of the fusion protein, a cell surface molecule which anchors the fusion protein to the cell membrane, and a detectable label such as a fluorescent protein or an enzyme.

In a specific embodiment of the invention, the nucleotide sequence of SGT4 could be synthesized in whole or in part, using chemical methods well known in the art. See, for example, Caruthers et al., 1980, Nuc. Acids Res. Symp. Ser. 7:215-233; Crea and Horn, 180, Nuc. Acids Res. 9(10):2331; Matteucci and Caruthers, 1980, Tetrahedron Letter 21:719; and Chow and Kempe, 1981, Nuc. Acids Res. 9(12):2807-2817. Alternatively, the polypeptide itself could be produced using chemical methods to synthesize an SGT4 amino acid sequence in whole or in part. For example, peptides can be synthesized by solid phase techniques, cleaved from the resin, and purified by preparative high performance liquid chromatography. (e.g., see Creighton, 1983, Proteins Structures And Molecular Principles, W. H. Freeman and Co., N.Y. pp.50-60). The composition of the synthetic peptides may be confirmed by amino acid analysis or sequencing (e.g., the Edman degradation procedure; see Creighton, 1983, Proteins, Structures and Molecular Principles, W.H. Freeman and Co., N.Y., pp.34-49).

In a specific embodiment of the invention, a polypeptide containing at least 10 (continuous) amino acids of the SGT4 protein is provided. In other embodiments, the polypeptide may contain at least 20 or 50 amino acids. In specific embodiments, such polypeptides do not contain more than 100, 150 or 200 amino acids. Derivatives or analogs of the polypeptides include, but are not limited to, molecules containing regions that are substantially homologous to the SGT4 protein or fragments thereof (e.g., in various embodiments, at least 60% or 70% or 80% or 90% or 95% identity over an amino acid sequence of identical size or when compared to an aligned sequence in which the alignment is done by a computer homology program known in the art) or product encoded by a polynucleotide that is capable of hybridizing to a naturally-occurring coding sequence, under highly stringent, moderately stringent, or low stringent conditions. Percent homolgy may be determined, for example, by comparing sequence information using the BLAST or GAP programs described supra.

The present invention also encompasses SGT4 polypeptides that are coded for by alternatively spliced SGT4 mRNA transcripts.

The derivatives and analogs of SGT4 protein can be produced by various methods known in the art. The manipulations which result in their production can occur at the nucleic acid or protein level. For example, a cloned coding sequence can be modified by any of numerous strategies known in the art (Maniatis, T., 1990, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of a polynucleotide encoding a derivative or analog, care should be taken to ensure that the modified coding sequence remains within the same translational reading frame as the antigen, uninterrupted by translational stop signals, in the coding region where the functional domain is encoded.

Additionally, the coding sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, chemical mutagenesis, in vitro site-directed mutagenesis (Hutchinson, C., et al., 1978, J. Biol. Chem 253:6551), use of TABS linkers (Pharmacia), and the like.

Manipulations may also be made at the protein level. Included within the scope of the invention are protein fragments or other derivatives or analogs which are differentially modified during or after translation, e.g., by glycosylation, acetylation, phosphorylation, amidation, derivatization by known protecting/blocking groups, proteolytic cleavage, linkage to a heterologous polypeptide or another antigen. Any of numerous chemical modifications may be carried out by known techniques, including but not limited to, specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

In addition, analogs and derivatives can be chemically synthesized. Non-classical amino acids (i.e., amino acids not encoded by the genetic code) or chemical amino acid analogs can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, omithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

In a specific embodiment, the derivative is a chimeric or fusion protein containing SGT4 or a fragment thereof joined at its amino- or carboxy-terminus to a heterologous protein via a peptide bond. Alternatively, the proteins are connected by a flexible polylinker such as Gly-Cys-Gly or Gly-Gly-Gly-Gly-Ser repeated 1 to 3 times (Bird et al., 1988, Science 242:423-426; Chaudhary et al., 1990, Proc. Nat'l. Acad. Sci. U.S.A. 87:1066-1070). In one embodiment, such a chimeric protein is produced by recombinant expression of a nucleic acid encoding the protein (an SGT4 coding sequence joined in-frame to a coding sequence for another antigen or a heterologous protein). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. Chimeric genes comprising portions of the SGT4 coding sequence fused to any other coding sequences may be constructed.

The invention further encompasses soluble protein or peptide derivatives of membrane-associated forms of SGT4. Such soluble derivatives can be engineered by excising the membrane-anchoring region of the protein, e.g., creating a derivative of SGT4 lacking a transmembrane domain, comprising only an extracellular and/or intracellular domain. Such soluble derivatives can bind to ligands of the full-length membrane associated protein, and can be used as therapeutic agents in a variety of contexts. For example, the soluble derivatives can compete with the membrane-associated form for ligand binding, thereby reducing the effective levels and biological activity of the ligand.

In another specific embodiment of the invention, the SGT4 derivative is a protein or peptide generated by some form of directed evolution, as described in Section 5.1. Such variants can possess enhanced or altered functional activity relative to naturally-occurring SGT4.

In another specific embodiment, the derivative is a molecule comprising a region of homology with SGT4. By way of example, in various embodiments, a protein region can be considered “homologous” to a second protein region when the amino acid sequence of the first region is at least 30%, 40%, 50%, 60%, 70%, 75%, 80%, 90%, or 95% identical, when compared to any sequence in the second region of an equal number of amino acids as the number contained in the first region or when compared to an aligned sequence of the second region that has been aligned by a computer homology program known in the art e.g., the BLAST program described above.

5.3. Production of SGT4 Polypeptides

In order to produce a biologically active SGT4, the nucleotide sequence coding for SGT4, or a functional equivalent, is inserted into an appropriate expression vector, i.e., a vector which contains the necessary elements for the transcription and translation of the inserted coding sequence. The SGT4 gene product as well as host cells or cell lines transfected or transformed with recombinant SGT4 gene-containing expression vectors can be used for a variety of purposes. These include, but are not limited to, large scale production of SGT4 protein, use of SGT4 as immunogen for antibody generation and screening of compounds that bind SGT4.

Methods which are well known to those skilled in the art can be used to construct expression vectors containing the SGT4 coding sequence and appropriate transcriptional/translational control signals. These methods include in vitro recombinant DNA techniques, synthetic techniques and in vivo recombination/genetic recombination. (See, for example, the techniques described in Sambrook et al., 1989, Molecular Cloning A Laboratory Manual, Cold Spring Harbor Laboratory, N.Y. and Ausubel et al., 1989, Current Protocols in Molecular Biology, Greene Publishing Associates and Wiley Interscience, N.Y.). RNA-capable of encoding SGT4 polypeptide may also be chemically synthesized (Gait, ed., 1984, Oligonucleotide Synthesis, IRL Press, Oxford).

A variety of host-expression vector systems may be utilized to express the SGT4 coding sequence. These include, but are not limited to, microorganisms such as bacteria (e.g., E. coli, B. subtilis) transformed with recombinant bacteriophage DNA, plasmid DNA or cosmid DNA expression vectors containing the SGT4 coding sequence; yeast (e.g., Saccharomyces, Pichia) transformed with recombinant yeast expression vectors containing the SGT4 coding sequence; insect cell systems infected with recombinant virus expression vectors (e.g., baculovirus) containing the SGT4 coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the SGT4 coding sequence; or mammalian cell systems (e.g., COS, CHO, BHK, 293, 3T3 cells). The expression elements of these systems vary in their strength and specificities.

Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter; cytomegalovirus promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll α/β binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the SGT4 coding sequence, SV40-, BPV- and EBV-based vectors may be used with an appropriate selectable marker.

5.3.1. Expression Systems

In bacterial systems a number of expression vectors may be advantageously selected depending upon the use intended for the expressed SGT4 product. For example, when large quantities of SGT4 protein are to be produced for the generation of antibodies, screening peptide libraries or formulating pharmaceutical compositions, vectors which direct the expression of high levels of fusion protein products that are readily purified may be desirable. Such vectors include but are not limited to the E. coli expression vector pUR278 (Ruther et al., 1983, EMBO J. 2:1791), in which the SGT4 coding sequence may be ligated into the vector in frame with the lacZ coding region so that a hybrid protein is produced; pIN vectors (Inouye & Inouye, 1985, Nucleic acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned polypeptide of interest can be released from the GST moiety.

In yeast, a number of vectors containing constitutive or inducible promoters may be used (Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol.152, pp.673-684; and The Molecular Biology of the Yeast Saccharomyces, 1982, Eds. Strathem et al., Cold Spring Harbor Press, Vols. I and II).

In cases where plant expression vectors are used, the expression of the SGT4 coding sequence may be driven by any of a number of promoters. For example, viral promoters such as the 35S RNA and 19S RNA promoters of CAMV (Brisson et al., 1984, Nature 310:511-514), or the coat protein promoter of TMV (Takamatsu et al., 1987, EMBO J. 6:307-311) may be used; alternatively, plant promoters such as the small subunit of RUBISCO (Coruzzi et al., 1984, EMBO J. 3:1671-1680; Broglie et al., 1984, Science 224:838-843); or heat shock promoters, e.g., soybean hsp17.5-E or hsp17.3-B (Gurley et al., 1986, Mol. Cell. Biol. 6:559-565) may be used. These constructs can be introduced into plant cells using Ti plasmids, Ri plasmids, plant virus vectors, direct DNA transformation, microinjection, electroporation, etc. (Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9).

An alternative expression system which can be used to express SGT4 is an insect system. In one such system, Autographa californica nuclear polyhedrosis virus (AcNPV) is used as a vector to express foreign genes. The virus grows in Spodoptera frugiperda cells. The SGT4 coding sequence may be cloned into non-essential regions (for example the polyhedron gene) of the virus and placed under control of an AcNPV promoter (for example the polyhedron promoter). Successful insertion of the SGT4 coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (see, e.g., Smith et al., 1983, J. Viol. 46:584; Smith, U.S. Pat. No. 4,215,051).

In mammalian host cells, a number of viral based expression systems may be utilized. In cases where an adenovirus is used as an expression vector, the SGT4 coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing SGT4 in infected hosts (e.g., See Logan & Shenk, 1984, Proc. Natl. Acad. Sci. USA 81:3655-3659). Alternatively, a vector derived from vaccinia virus can be used, which would typically make use of the vaccinia 7.5K promoter (See, e.g., Mackett et al., 1982, Proc. Natl. Acad. Sci. USA 79:7415-7419; Mackett et al., 1984, J. Virol. 49:857-864; Panicali et al., 1982, Proc. Natl. Acad. Sci. USA 79:4927-4931). Regulatable expression vectors such as the tetracycline repressible vectors may also be used to express the coding sequences in a controlled fashion.

Specific initiation signals may also be required for efficient translation of inserted SGT4 coding sequences. These signals include the ATG initiation codon and adjacent sequences. In cases where the entire SGT4 gene, including its own initiation codon and adjacent sequences, is inserted into the appropriate expression vector, no additional translational control signals may be needed. However, in cases where only a portion of the SGT4 coding sequence is inserted, exogenous translational control signals, including the ATG initiation codon, may need to be provided. Furthermore, the initiation codon must be in phase with the reading frame of the SGT4 coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, WI38, yolk sac cells, etc.

For long-term, high-yield production of recombinant proteins, stable expression is preferred. For example, cell lines which stably express the SGT4 protein may be engineered. Rather than using expression vectors which contain viral origins of replication, host cells can be transformed with the SGT4 coding sequence controlled by appropriate expression control elements (e.g., promoter and/or enhancer sequences, transcription terminators, polyadenylation sites, etc.), and a selectable marker. Following the introduction of foreign DNA, genetically engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the SGT4 protein. Such engineered cell lines are particularly useful in screening for molecules or drugs that affect SGT4 function.

A number of selectable markers may be used, including but not limited to, the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in cells that do not express the selectable marker endogenously. Also, antimetabolite resistance can be used as the basis of selection, using a selectable marker gene such as dhfr, which confers resistance to methotrexate (Wigler, et al., 1980, Proc. Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); or hygro, which confers resistance to hygromycin (Santerre, et al., 1984, Gene 30:147). Other selectable markers include the genes trpB, which allows cells to utilize indole in place of tryptophan; hisD, which allows cells to utilize histinol in place of histidine (Hartman & Mulligan, 1988, Proc. Natl. Acad. Sci. USA 85:8047); ODC (omithine decarboxylase) which confers resistance to the omithine decarboxylase inhibitor, 2-(difluoromethyl)-DL-omithine, DFMO (McConlogue L., 1987, In: Current Communications in Molecular Biology, Cold Spring Harbor Laboratory ed.) and the glutamine synthetase gene (Bebbington et al., 1992, Biotech 10:169).

The expression characteristics of an endogenous SGT4 gene within a cell line or microorganism may be modified by inserting a heterologous DNA regulatory element into the genome of a stable cell line or cloned microorganism such that the inserted regulatory element is operatively linked with the endogenous SGT4 gene. For example, an endogenous SGT4 gene which is normally “transcriptionally silent”, i.e., an SGT4 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous SGT4 gene may be activated by insertion of a promiscuous regulatory element that works across cell types.

A heterologous regulatory element may be inserted into a stable cell line or cloned microorganism, such that it is operatively linked with an endogenous SGT4 gene, using techniques which are well known to those of skill in the art, such as targeted homologous recombination (e.g., in Chappel, U.S. Pat. No. 5,272,071; PCT publication No. WO 91/06667, published May 16, 1991).

5.3.2. Protein Purification

Once a recombinant protein is expressed, it can be identified by assays based on the physical or functional properties of the product, including radioactive labeling of the product followed by analysis by gel electrophoresis, radioimmunoassay, ELISA, bioassays, etc.

Once the encoded protein is identified, it may be isolated and purified by standard methods including chromatography (e.g., high performance liquid chromatography, ion exchange, affinity, and sizing column chromatography), centrifugation, differential solubility, or by any other standard technique for the purification of proteins. The actual conditions used will depend, in part, on factors such as net charge, hydrophobicity, hydrophilicity, etc., and will be apparent to those having skill in the art. The functional properties may be evaluated using any suitable assay, e.g. an assay for the ability to activate a GTPase. For the practice of the present invention, it is preferred that the polypeptide is at least 80% purified from other proteins. It is more preferred that they are at least 90% purified. For in vivo administration, it is preferred that it is greater than 95% purified, and more preferably greater than 99% purified.

In another alternate embodiment, native proteins can be purified from natural sources, by standard methods such as those described above (e.g., immunoaffinity purification). In a specific embodiment of the present invention, the SGT4 polypeptides, whether produced by recombinant DNA techniques or by chemical synthetic methods or by purification from natural sources include, but are not limited to, those containing, as a primary amino acid sequence, all or part of the amino acid sequences substantially as recited in FIGS. 2 and 4, as well as fragments and other derivatives, and analogs thereof, including proteins homologous thereto.

5.4. Identification of Cells that Express SGT4

The host cells which contain the coding sequence and which express an SGT4 gene product, fragments thereof, or an SGT4 fusion protein may be identified by at least four general approaches; (a) DNA-DNA or DNA-RNA hybridization; (b) the presence or absence of “marker” gene functions; (c) assessing the level of transcription as measured by the expression of SGT4 mRNA transcripts in the host cell; and (d) detection of the gene product as measured by its biological activity or by use of analytical techniques such mass-spectroscopy, amino acid sequencing or immunodetection. Prior to the identification of gene expression, the host cells may be first mutagenized in an effort to increase the level of expression of SGT4, especially in cell lines that produce low amounts of SGT4.

In the first approach, the presence of the SGT4 coding sequence inserted in the expression vector can be detected by DNA-DNA or DNA-RNA hybridization using probes comprising nucleotide sequences that are homologous to the SGT4 coding sequence or portions or derivatives thereof.

In the second approach, the recombinant expression vector/host system can be identified and selected based upon the presence or absence of certain “marker” gene functions (e.g., thymidine kinase activity, resistance to antibiotics, resistance to methotrexate, transformation phenotype, occlusion body formation in baculovirus, etc.). For example, if the SGT4 coding sequence is inserted within a marker gene sequence of the vector, recombinants containing the SGT4 coding sequence can be identified by the absence of the marker gene function. Alternatively, a marker gene can be placed in tandem with the SGT4 coding sequence under the control of the same or different promoter used to control the expression of the SGT4 coding sequence. Expression of the marker in response to induction or selection indicates expression of the SGT4 coding sequence.

In the third approach, transcriptional activity for the SGT4 coding region can be assessed by hybridization assays. For example, RNA can be isolated and analyzed by Northern blot using a probe homologous to the SGT4 coding sequence or particular portions thereof. Alternatively, total nucleic acids of the host cell may be extracted and assayed for hybridization to such probes. Additionally, RT-PCR may be used to detect low levels of gene expression.

In the fourth approach, the expression of the SGT4 protein product can be assessed immunologically, for example by Western blots, immunoassays such as radioimmuno-precipitation, enzyme-linked immunoassays and the like. This can be achieved by using an anti-SGT4 antibody. Expression of the SGT4 protein product can also be assessed using analytical techniques such as amino acid sequencing, which can be accomplished by means of, for example, Edman degradation or tandem mass spectroscopy, or by analysis of the masses of peptides generated by partial hydrolysis of the protein product using mass spectroscopy. In the identification of SGT4 protein by mass spectroscopy, it will often be desirable to separate the SGT4 protein from other protein constituents of the cell by means of two-dimensional gel electrophoresis, partially hydrolyze the isolated protein using an amino acid specific protease (e.g., Lys-C, trypsin), and then determine the mass of the resulting peptide fragments using mass spectroscopy. Determination of peptide mass can then be used to identify the protein as SGT4, or a variant thereof, using a database of the predicted masses of protein proteolysis products and analysis software such as Protein Prospector, which is publicly available on the internet at http://prospector.ucsf.edu.

5.5. Antibodies to SGT4 And Their Uses

Antibodies directed to SGT4 are useful for, among other things, the identification and isolation of SGT4. In a preferred embodiment, an anti-SGT4 antibody binds and/or competitively inhibits SGT4 protein and neutralize its activity, thereby reducing the effective levels of the protein in the body. Alternatively, an anti-SGT4 antibody may activate SGT4 function. Anti-SGT4 antibodies may be used in detecting and quantifying expression of SGT4 levels in cells and tissues such as endothelial cells and certain tumor cells, as well as isolating SGT4-positive cells from a cell mixture or eliminating such cells by means of immunotoxins.

Various procedures known in the art may be used for the production of antibodies to epitopes of the naturally-occurring, synthetic and recombinantly produced SGT4 protein. Such antibodies include, but are not limited, to polyclonal, monoclonal, chimeric, humanized, single chain, anti-idiotypic, antigen-binding antibody fragments and fragments produced by a variable region expression library. Neutralizing antibodies, i.e., those which compete for the substrate binding site and/or ligand binding domain of the SGT4 protein are also encompassed by the invention. In a therapeutic embodiment of the invention, neutralizing antibodies that bind to membrane associated proteins can be used to either mimic the natural ligand or block the binding of the natural ligand, e.g., as an agonist or antagonist.

Monoclonal antibodies that bind SGT4 may be radioactively labeled, thereby allowing one to follow their location and distribution in the body after injection. Radioisotope tagged antibodies may be used as a non-invasive diagnostic tool for imaging de novo endothelial cells in tumors and metastases.

Immunotoxins may also be designed which target cytotoxic agents to specific sites in the body. For example, high affinity SGT4-specific monoclonal antibodies may be covalently complexed to bacterial or plant toxins, such as diphtheria toxin or ricin. A general method of preparation of antibody/hybrid molecules may involve use of thiol-crosslinking reagents such as SPDP, which attack the primary amino groups on the antibody and by disulfide exchange, attach the toxin to the antibody. The hybrid antibodies may be used to specifically eliminate SGT4-expressing cells or tissues in tumors.

For the production of antibodies, various host animals may be immunized by injection with the recombinant or naturally purified SGT4 protein, fusion protein or peptides, including but not limited to rabbits, mice, rats, hamsters, and the like. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to SGT4 may be prepared by using any technique which provides for the production of antibody molecules by continuous cell lines in culture. These include but are not limited to the hybridoma technique originally described by Kohler and Milstein, (Nature, 1975, 256:495-497), the human B-cell hybridoma technique (Kosbor et al., 1983, Immunology Today, 4:72; Cote et al., 1983, Proc. Natl. Acad. Sci., 80:2026-2030) and the EBV-hybridoma technique (Cole et al., 1985, Monoclonal Antibodies and Cancer Therapy, Alan R. Liss, Inc., pp. 77-96). Such antibodies may be of any immunoglobulin class including, but not limited to, IgG, IgM, IgE, IgA, IgD and any subclass thereof. The hybridoma producing the monoclonal antibodies of this invention may be cultivated in vitro or in vivo.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (11987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Fremont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

In a preferred embodiment of the invention, monoclonal antibodies to SGT4 can be used as therapeutic agents. For example, such monoclonal antibodies can be used to reduce the effective level of SGT4 or related proteins in the body, or to either mimic or block the binding of a natural ligand of SGT4, particularly ligands of membrane-associated forms of SGT4. The use of humanized monoclonal antibodies is preferred in most human therapeutic applications. Humanized antibodies can be generated, for example, using techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454; U.S. Pat. Nos. 4,816,567 and 4,816,397) by splicing the genes from a mouse antibody molecule of appropriate antigen specificity together with genes from a human antibody molecule of appropriate biological activity. Humanized antibodies may also be generated according to the methods described in U.S. Pat. Nos. 5,693,762; 5,585,089 and 5,565,332.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce single chain antibodies against gene products of interest. Single chain antibodies are formed by linking the heavy and light chain fragments of the Fv region via an amino acid bridge, resulting in a single chain polypeptide.

Antibodies to the polypeptides of the invention can, in turn, be utilized to generate anti-idiotype antibodies that mimic an epitope of the polypeptide of interest, using techniques well known to those skilled in the art. (See, e.g., Greenspan & Bona, 1993, FASEB J 7(5):437-444; and Nissinoff, 1991, J. Immunol. 147(8):2429-2438). For example, antibodies which competitively inhibit the binding of an antibody to an antigenic peptide may mimic the antigenic epitope of the peptide. Such neutralizing anti-idiotypes or Fab fragments of such anti-idiotypes can be used.

Hybridomas may be screened using enzyme-linked immunosorbent assays (ELISA) or radioimmunoassays in order to detect cultures secreting antibodies specific for refolded recombinant SGT4. Subsequent testing may use recombinant SGT4 fragments to identify the specific portion of the SGT4 molecule with Which a monoclonal antibody binds. Additional testing may be used to identify monoclonal antibodies with desired functional characteristics such as staining of histological sections, immunoprecipitation or Western blotting of SGT4, or neutralization of SGT4 activity. Determination of the monoclonal antibody isotype may be accomplished by ELISA, thus providing additional information concerning purification or function.

Antibody fragments which recognize specific binding sites of SGT4 may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281; U.S. Pat. Nos. 5,223,409, 5,403,484 and 5,571,698) to allow rapid and easy identification of monoclonal Fab fragments with the desired specificity to SGT4. Antibody constant regions can be altered by molecular manipulations to modify their effector functions (U.S. Pat. No. 5,624,821). The complementarity-determining regions (CDR) of an antibody can be identified, and synthetic peptides corresponding to such regions are used to mediate antigen binding (U.S. Pat. No. 5,637,677).

5.6 Research uses of the Present Invention

The polynucleotides, proteins, antibodies, vectors, host cells, and other aspect of the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

5.7 Nutritional Uses of the Present Invention

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

5.8 Assays for Proteins That Interact with SGT4

Any method suitable for detecting protein-protein interactions may be employed for identifying proteins, including but not limited to transmembrane or intracellular proteins that interact with SGT4. Among the traditional methods which may be employed are co-immunoprecipitation, crosslinking and co-purification through gradients or chromatographic columns to identify proteins in that interact with SGT4. For such assays, the SGT4 component can be a full length protein, a soluble derivative thereof, a peptide corresponding to domain of interest, or a fusion protein containing some region of SGT4.

Methods may be employed which result in the simultaneous identification of genes that encode proteins capable of interacting SGT4. These methods include, for example, probing expression libraries, in a manner similar to the well known technique of antibody probing of λgt11 libraries, using labeled SGT4 or a variant thereof.

One method which detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to a nucleotide sequence encoding SGT4, or a polypeptide, peptide, or fusion protein therefrom, and the other plasmid consists of nucleotides encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, SGT4 can be used as the bait gene product. Total genomic or αDNA sequences are fused to the DNA encoding an activation domain. This library and a plasmid encoding a hybrid of a bait SGT4 gene product fused to the DNA-binding domain are cotransformed into a yeast reporter strain, and the resulting transformants are screened for those that express the reporter gene. For example, and not by way of limitation, a bait SGT4 gene sequence, e.g., the genes open reading frame, can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait the SGT4 gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait SGT4 gene-GAL4 fusion plasmid into a yeast strain which contains a lacZ gene driven by a promoter which contains GAL4 activation sequence. A cDNA encoded protein, fused to GAL4 transcriptional activation domain, that interacts with the bait SGT4 gene product will reconstitute an active GAL4 protein and thereby drive expression of the HIS3 gene. Colonies which express HIS3 can be detected by their growth on petri dishes containing semi-solid agar based media lacking histidine. The cDNA can then be purified from these strains, and used to produce and isolate the bait SGT4 gene-interacting protein using techniques routinely practiced in the art.

5.9 Screening Assays for Compounds That Modulate SGT4 Expression or Activity

The following assays are designed to identify compounds that interact with (e.g., bind to) SGT4, compounds that interfere with the interaction of SGT4 with its ligand binding partner, cognate or substrate, and to compounds that modulate the activity of SGT4 gene expression (i.e., modulate the level of SGT4 gene expression) or modulate the levels of SGT4 in the body. Assays may additionally be utilized which identify compounds that bind to SGT4 gene regulatory sequences (e.g., promoter sequences) and, consequently, may modulate SGT4 gene expression. See, e.g., Platt, K. A., 1994, J. Biol. Chem. 269:28558-28562, which is incorporated herein by reference in its entirety.

The compounds which may be screened in accordance with the invention include but are not limited to peptides, antibodies and fragments thereof, and other organic compounds (e.g., peptidomimetics) that bind to a SGT4 and either mimic the activity triggered by a natural ligand (i.e., agonists) or inhibit the activity triggered by the natural ligand (i.e., antagonists); as well as peptides, antibodies or fragments thereof, and other organic compounds that mimic SGT4 (or a portion thereof) and bind to and “activate” or “neutralize” the natural ligand or substrate.

Such compounds may include, but are not limited to, peptides such as, for example, soluble peptides, including but not limited to members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Other compounds which can be screened in accordance with the invention include but are not limited to small organic molecules that are able to cross the blood-brain barrier, gain entry into an appropriate cell (e.g., in the choroid plexus, pituitary, the hypothalamus, etc.) and affect the expression of an SGT4 gene or some other gene involved in an SGT4 mediated pathway (e.g., by interacting with the regulatory region or transcription factors involved in gene expression); or such compounds that affect or substitute for the activity of the SGT4 or the activity of some other intracellular factor involved in a SGT4 signal transduction, catabolic, or metabolic pathways.

Computer modeling and searching technologies permit identification of compounds, or the improvement of already identified compounds, that can modulate SGT4 expression or activity. Having identified such a compound or composition, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site (or binding site), either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential modulators of SGT4 activity.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites (or binding sites) of an SGT4, and related transduction and transcription factors will be apparent to those of skill in the art.

Examples of molecular modeling systems are the CHARMm and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236:125-140 and 141-162; and, with respect to a model receptor for nucleic acid components, Askew, et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators.

Compounds identified via assays such as those described herein may be useful, for example, in elucidating the biological function of a SGT4 gene product. Such compounds can be administered to a patient at therapeutically effective doses to treat any of a variety of physiological or mental disorders. A therapeutically effective dose refers to that amount of the compound sufficient to result in any amelioration, impediment, prevention, or alteration of any biological symptom.

5.9.1. Screening Assays for Compounds That Bind to SGT4

Systems may be designed to identify compounds capable of interacting with (e.g., binding to) or mimicking SGT4, or capable of interfering with the binding of SGT4 to a cognate ligand, binding partner or substrate. The compounds identified can be useful, for example, in modulating the activity of wild type and/or mutant SGT4 gene products; can be useful in elaborating the biological function of SGT4; can be utilized in screens for identifying compounds that disrupt normal SGT4 interactions; or may themselves disrupt or activate such interactions.

The principle of the assays used to identify compounds that bind to SGT4, or SGT4 cognate ligands or substrates, involves preparing a reaction mixture of SGT4 and the test compound under conditions and for a time sufficient to allow the two components to interact and bind, thus forming a complex which can be removed and/or detected in the reaction mixture. The SGT4 species used can vary depending upon the goal of the screening assay. For example, where agonists of the natural receptor are desired, the full length SGT4, or a soluble truncated SGT4, a peptide, or fusion protein containing one or more SGT4 domains fused to a protein or polypeptide that affords advantages in the assay system (e.g., labeling, isolation of the resulting complex, etc.) can be utilized. Where compounds that directly interact with SGT4 are sought, peptides corresponding to the SGT4 and fusion proteins containing SGT4 can be used.

The screening assays can be conducted in a variety of ways. For example, one method to conduct such an assay would involve anchoring the SGT4, polypeptide, peptide, or fusion protein therefrom, or the test substance onto a solid phase and detecting SGT4/test compound complexes anchored on the solid phase at the end of the reaction. In one embodiment of such a method, the SGT4 reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for a SGT4 protein, polypeptide, peptide or fusion protein or the test compound to anchor any complexes formed in solution, and a labeled antibody specific for the other component of the possible complex to detect anchored complexes.

5.9.2. Assays for Compounds That Interfere with Interactions Involving SGT4

Macromolecules that interact with SGT4 are referred to, for purposes of this discussion, as “binding partners”. These binding partners are likely to be involved in the SGT4 mediated biological pathways. Therefore, it is desirable to identify compounds that interfere with or disrupt the interaction of such binding partners which may be useful in regulating or augmenting SGT4 activity in the body and/or controlling disorders associated with this activity (or a deficiency thereof).

The basic principle of the assay systems used to identify compounds that interfere with the interaction between SGT4 and its binding partner or partners involves preparing a reaction mixture containing SGT4, or some variant thereof, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the SGT4 and its binding partner. Control reaction mixtures are incubated without the test compound or with a placebo. The formation of any complexes between the SGT4 and the binding partner is then detected. The formation of a complex in the control reaction, but not in the reaction mixture containing the test compound, indicates that the compound interferes with the interaction of the SGT4 and the interactive binding partner. Additionally, complex formation within reaction mixtures containing the test compound and normal SGT4 protein may also be compared to complex formation within reaction mixtures containing the test compound and a mutant SGT4. This comparison may be important in those cases wherein it is desirable to identify compounds that specifically disrupt interactions of mutant, or mutated, SGT4 but not the normal proteins.

The assay for compounds that interfere with the interaction between SGT4 and binding partners can be conducted in a heterogeneous or homogeneous format. Heterogeneous assays involve anchoring either the SGT4, or the binding partner, onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to, or simultaneously with, SGT4 and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either SGT4 or an interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the SGT4 or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of SGT4 and an interactive binding partner is prepared in which either the SGT4 or its binding partners is labeled, but the signal generated by the label is quenched due to formation of the complex (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt the interaction can be identified.

In a particular embodiment, a SGT4 fusion can be prepared for immobilization. For example, SGT4, or a peptide fragment thereof, can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art and described above. This antibody can be labeled with the radioactive isotope ¹²⁵I, for example, by methods routinely practiced in the art. In a heterogeneous assay, the fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between SGT4 and the interactive binding partner can be detected by measuring the amount of radioactivity that remains associated with the glutathione-agarose beads. A successful inhibition of the interaction by the test compound will result in a decrease in measured radioactivity.

Alternatively, the GST fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the interaction between SGT4 and the binding partner can be detected by adding the labeled antibody and measuring the radioactivity associated with the beads.

In another embodiment of the invention, these same techniques can be employed using peptide fragments that correspond to the binding domains of SGT4 and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensatory mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a relatively short, labeled peptide comprising the binding domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

For example, and not by way of limitation, a SGT4 can be anchored to a solid material as described, above, by making a GST fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored fusion protein and allowed to bind. After washing away unbound peptides, labeled bound material, representing the intracellular binding partner binding domain, can be eluted, purified, and analyzed for amino acid sequence by well-known methods. Peptides so identified can be produced synthetically or fused to appropriate facilitative proteins using recombinant DNA technology.

Cell-based systems can also be used to identify compounds that bind (or mimic) SGT4, or interfere with the binding of SGT4 to a binding partner or substrate. Such systems can be used to assess the altered activity associated with such binding in living cells. One tool of particular interest for such assays is green fluorescent protein which is described, inter alia, in U.S. Pat. No. 5,625,048, herein incorporated by reference. Cells that may be used in such cellular assays include, but are not limited to, leukocytes, or cell lines derived from leukocytes, lymphocytes, stem cells, including embryonic stem cells, and the like. In addition, expression host cells (e.g., B95 cells, COS cells, CHO cells, OMK cells, fibroblasts, Sf9 cells) genetically engineered to express a functional SGT4 of interest and to respond to activation by the test, or natural, ligand, as measured by a chemical or phenotypic change, or induction of another host cell gene, can be used as an end point in the assay.

5.10. Uses of Genetically Engineered Host Cells

In an embodiment of the invention, the SGT4 protein and/or cell lines that express SGT4 may be used to screen for antibodies, peptides, small molecules, natural and synthetic compounds or other cell bound or soluble molecules that bind to the SGT4 protein, especially those that cause a stimulation or inhibition of SGT4 function. Such compounds will typically be capable of binding to an active site, ligand binding site, or other functional domain of the SGT4 protein, thereby affecting the biological activity of the protein. For example, anti-SGT4 antibodies may be used to inhibit or stimulate SGT4 function and to detect its presence. Alternatively, screening of peptide libraries with recombinantly expressed soluble SGT4 protein or cell lines expressing SGT4 protein may be useful for identification of therapeutic molecules that function by inhibiting or stimulating the biological activities of SGT4. The uses of the SGT4 protein and engineered cell lines, described in the sections below, may be employed equally well for homologous SGT4 genes in various species.

In one embodiment of the invention, engineered cell lines which express the SGT4 coding region or a portion of it that is fused to another molecule such as the immunoglobulin constant region (Hollenbaugh and Aruffo, 1992, Current Protocols in Immunology, Unit 10.19; Aruffo et al., 1990, Cell 61:1303) may be utilized to produce a soluble molecule with increased half life. The soluble protein or fusion protein may be used in binding assays, affinity chromatography, immunoprecipitation, Western blot, and the like. Synthetic compounds, natural products, and other sources of potentially biologically active materials can be screened in assays that are well known in the art.

Random peptide libraries consisting of all possible combinations of amino acids attached to a solid phase support may be used to identify peptides that are able to bind to SGT4, especially its active site (Lam, K. S. et al., 1991, Nature 354: 82-84). The screening of peptide libraries may have therapeutic value in the discovery of pharmaceutical agents that stimulate or inhibit the biological activities of SGT4.

Identification of molecules that are able to bind to the SGT4 protein may be accomplished by screening a peptide library with recombinant soluble SGT4 protein. Methods for expression and purification of SGT4 are described in Section 5.3, and may be used to express recombinant full length SGT4 or fragments of SGT4 depending on the functional domains of interest. SGT4 may be used to identify a cofactor such as apolipoprotein.

To identify and isolate the peptide/solid phase support that interacts and forms a complex with SGT4, it may be necessary to label or “tag” the SGT4 molecule. In addition, anti-SGT4 antibody may be used to detect SGT4 bound to a second molecule. The SGT4 protein may be conjugated to enzymes such as alkaline phosphatase or horseradish peroxidase or to other reagents such as fluorescent labels which may include fluorescein isothiocyanate (FITC), phycoerythrin (PE) or rhodamine. Conjugation of any given label to SGT4 may be performed using techniques that are well known in the art. Alternatively, SGT4-containing expression vectors may be engineered to express a chimeric SGT4 protein containing an epitope for which a commercially available antibody exist. The epitope specific antibody may be tagged using methods well known in the art including labeling with enzymes, fluorescent dyes or colored or magnetic beads.

The “tagged” SGT4 conjugate is incubated with the random peptide library for 30 minutes to one hour at 22° C. to allow complex formation between SGT4 and peptide species within the library. The library is then washed to remove any unbound protein. If SGT4 has been conjugated to alkaline phosphatase or horseradish peroxidase the whole library is poured into a petri dish containing substrates for either alkaline phosphatase or peroxidase, for example, 5-bromo-4-chloro-3-indoyl phosphate (BCIP) or 3,3′,4,4″-diaminobenzidine (DAB), respectively. After incubating for several minutes, the peptide/solid phase-SGT4 complex changes color, and can be easily identified and isolated physically under a dissecting microscope with a micromanipulator. If a fluorescent tagged SGT4 molecule has been used, complexes may be isolated by fluorescence activated sorting. If a chimeric SGT4 protein expressing a heterologous epitope has been used, detection of the peptide/SGT4 complex may be accomplished by using a labeled epitope specific antibody. Once isolated, the identity of the peptide attached to the solid phase support may be determined by peptide sequencing.

In addition to using soluble SGT4 molecules, it is possible to detect peptides that bind to cell-associated SGT4 using intact cells. The use of intact cells is preferred for use with cell surface molecules. Methods for generating cell lines expressing SGT4 are described in Section 5.3. The cells used in this technique may be either live or fixed cells. The cells may be incubated with the random peptide library and bind to certain peptides in the library to form a “rosette” between the target cells and the relevant solid phase support/peptide. The rosette can thereafter be isolated by differential centrifugation or removed physically under a dissecting microscope. Intracellular proteins can be accessed by treating the cells with detergent.

As an alternative to whole cell assays for membrane bound receptors or receptors that require the lipid domain of the cell membrane to be functional, SGT4 molecules can be reconstituted into liposomes where label or “tag” can be attached.

5.11. Uses of SGT4 Polynucleotide

An SGT4 polynucleotide may be used for diagnostic and/or therapeutic purposes, particularly with respect to conditions or diseases related to a signal transduction mechanism involving SGT4, e.g., signal transduction pathways regulated by GTP binding proteins. These signal transduction mechanisms regulate various aspects of cellular physiology, including cell survival, proliferation and differentiation, thus abnormalities in these mechanisms can lead to a variety of pathological or abnormal conditions. In addition, since SGT4 and their variants are expressed at higher levels in certain specific tissue and cell types, particularly neuronal tissue, heart, liver, pancreas and adrenal gland, an SGT4 polynucleotide may be used to detect the expression of SGT4 as markers of these specific cells and tissues. For diagnostic purposes, an SGT4 polynucleotide may be used to detect the level of SGT4 gene expression, aberrant SGT4 gene expression or mutations in disease states. Included in the scope of the invention are oligonucleotides such as antisense RNA and DNA molecules, and ribozymes, that function to inhibit translation of SGT4. An SGT4 polynucleotide may also be used to construct transgenic and knockout animals for studying SGT4 function in vivo and for the screening of SGT4 agonists and antagonists in an animal model.

5.11.1. Transgenic and Knockout Animals

The SGT4 gene products can be expressed in animals by transgenic technology. Animals of any species, including, but not limited to, mice, rats, rabbits, guinea pigs, pigs, micro-pigs, goats, sheep, and non-human primates, e.g., baboons, monkeys, and chimpanzees may be used to generate SGT4 transgenic animals. The term “transgenic,” as used herein, refers to animals expressing SGT4 coding sequences from a different species (e.g., mice expressing human SGT4 gene sequences), as well as animals that have been genetically engineered to overexpress endogenous (i.e., same species) SGT4 sequences or animals that have been genetically engineered to no longer express endogenous SGT4 gene sequences (i.e., “knock-out” animals), and their progeny.

Any technique known in the art may be used to introduce an SGT4 transgene into animals to produce the founder lines of transgenic animals. Such techniques include, but are not limited to, pronuclear microinjection (Hoppe and Wagner, 1989, U.S. Pat. No. 4,873,191); retrovirus-mediated gene transfer into germ lines (Van der Putten, et al., 1985, Proc. Natl. Acad. Sci., USA 82:6148-6152); gene targeting in embryonic stem cells (Thompson, et al., 1989, Cell 56:313-321); electroporation of embryos (Lo, 1983, Mol. Cell. Biol. 3:1803-1814); and sperm-mediated gene transfer (Lavitrano et al., 1989, Cell 57:717-723) (see Gordon, 1989, Transgenic Animals, Intl. Rev. Cytol. 115, 171-229).

Any technique known in the art may be used to produce transgenic animal clones containing an SGT4 transgene, for example, nuclear transfer into enucleated oocytes of nuclei from cultured embryonic, fetal or adult cells induced to quiescence (Campbell, et al., 1996, Nature 380:64-66; Wilmut, et al., 1997, Nature 385:810-813).

The present invention provides for transgenic animals that carry an SGT4 transgene in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (1992, Proc. Natl. Acad. Sci. USA 89:6232-6236). The regulatory sequences required for such a cell-type specific activation will depend upon the particular cell type of interest, and will be apparent to those of skill in the art. When it is desired that the SGT4 transgene be integrated into the chromosomal site of the endogenous SGT4 gene, gene targeting is preferred. Briefly, when such a technique is to be utilized, vectors containing some nucleotide sequences homologous to the endogenous SGT4 gene are designed for the purpose of integrating, via homologous recombination with chromosomal sequences, into and disrupting the function of the nucleotide sequence of the endogenous SGT4 gene. The transgene may also be selectively introduced into a particular cell type, thus inactivating the endogenous SGT4 gene in only that cell type, by following, for example, the teaching of Gu et al. (1994, Science 265: 103-106). The regulatory sequences required for such a cell-type specific inactivation will depend upon the particular cell type of interest, and will be apparent to those of skill in the art.

Once transgenic animals have been generated, the expression of the recombinant SGT4 gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include, but are not limited to, Northern blot analysis of tissue samples obtained from the animal, in situ hybridization analysis, and RT-PCR. Samples of SGT4 gene-expressing tissue, may also be evaluated immunocytochemically using antibodies specific for the SGT4 transgene product.

5.11.2. Diagnostic Uses of SGT4 Polynucleotide

An SGT4 polynucleotide may have a number of uses for the diagnosis of diseases resulting from aberrant expression of SGT4. Alternatively, polymorphisms or mutations may be identified in an SGT4 nucleotide sequence which may be correlative with disease. For example, the SGT4 nucleotide sequence or portions thereof may be used in hybridization assays of biopsies or autopsies to diagnose abnormalities of SGT4 expression; e.g., Southern analysis, Northern analysis, in situ hybridization assays and PCR. For PCR, primers of 15-25 nucleotides designed from any portion of SGT4 nucleotide sequence are preferred. However, the length of primers may be adjusted by one skilled in the art. Such techniques are well known in the art, and are in fact the basis of many commercially available diagnostic kits. In some cases the detection of decreased SGT4 expression or a mutation in SGT4 may be used to determine an underlying cause of a disease, and thereby facilitate treatment of the disease. For example, detection of decreased SGT4 expression or a mutation in SGT4 can be diagnostic for a disease involving the disruption or perturbation of a cellular signal transduction mechanism, particularly signal transduction pathways regulated by GTP-binding proteins.

5.11.3. Therapeutic Uses of SGT4 Polynucleotide

An SGT4 polynucleotide may be useful in the treatment of various abnormal conditions, particularly conditions involving signal transduction mechanisms. e.g., cancer. By introducing gene sequences into cells, gene therapy can be used to treat conditions in which the cells do not express normal SGT4 or express abnormal/inactive SGT4. In some instances, the polynucleotide encoding SGT4 is intended to replace or act in the place of a functionally deficient endogenous gene. Alternatively, abnormal conditions characterized by overexpression can be treated using the gene therapy techniques described below.

In a specific embodiment, nucleic acids comprising a sequence encoding an SGT4 protein or a functional derivative thereof, are administered to promote SGT4 function, by way of gene therapy. Gene therapy refers to therapy performed by the administration of a nucleic acid to a subject. In this embodiment of the invention, the nucleic acid produces its encoded protein that mediates a therapeutic effect by promoting SGT4 function. Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 199.1, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred embodiment of the invention, the therapeutic composition comprises an SGT4 coding sequence that is part of an expression vector. In particular, such a nucleic acid has a promoter operably linked to the SGT4 coding sequence, said promoter being inducible or constitutive, and, optionally, tissue-specific. In another specific embodiment, a nucleic acid molecule is used in which the SGT4 coding sequence and any other desired sequences are flanked by regions that promote homologous recombination at a desired site in the genome, thus providing for intrachromosomal expression of the SGT4 nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy. In a specific embodiment, the nucleic acid is directly administered in vivo, where it is expressed to produce the encoded product. This can be accomplished by any methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus, or by administering it in linkage to a ligand subject to receptor-mediated endocytosis (see e.g., Wu and Wu, 1987, J. Biol. Chem. 262:4429-4432) which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a preferred embodiment of the invention, adenoviruses as viral vectors can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells (Kozarsky and Wilson, 1993, Current Opinion in Genetics and Development 3:499-503). Bout et al., (1994, Human Gene Therapy 5:3-10) demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234. Adeno-associated virus (AAV) has also been proposed for use in gene therapy (Walsh et al., 1993, Proc. Soc. Exp. Biol. Med. 204:289-300).

In addition, retroviral vectors (see Miller et al., 1993, Meth. Enzymol. 217:581-599) have been modified to delete retroviral sequences that are not necessary for packaging of the viral genome and integration into host cell DNA. The SGT4 coding sequence to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Another approach to gene therapy involves transferring a gene to cells in tissue culture. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, lipofection, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, endothelial cells are injected, e.g., subcutaneously. In another embodiment, recombinant skin cells may be applied as a skin graft onto the patient. The amount of cells envisioned for use depends on the desired effect, patient state, etc., and can be determined by one skilled in the art.

Cells into which a nucleic acid can be introduced for purposes of gene therapy encompass any desired, available cell type, and include, but are not limited to, neuronal, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. In a preferred embodiment, the cell used for gene therapy is autologous to the patient.

In a specific embodiment, the nucleic acid to be introduced for purposes of gene therapy comprises an inducible promoter operably linked to the coding sequence, such that expression of the nucleic acid is controllable by controlling the presence or absence of the appropriate inducer of transcription.

Oligonucleotides such as anti-sense RNA and DNA molecules, and ribozymes that function to inhibit the translation of a SGT4 mRNA are also within the scope of the invention. Such molecules are useful in cases where downregulation of SGT4 expression is desired. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a SGT4 nucleotide sequence, are preferred.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 11-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Within the scope of the invention are engineered hammerhead motif ribozyme molecules that specifically and efficiently catalyze endonucleolytic cleavage of SGT4 RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Endogenous target gene expression can also be reduced by inactivating or “knocking out” the target gene or its promoter using targeted homologous recombination (e.g., see Smithies, et al., 1985, Nature 317:230-234; Thomas and Capecchi, 1987, Cell 51:503-512; Thompson, et al., 1989, Cell 5:313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in experiments where modifications to ES (embryonic stem) cells can be used to generate animal offspring with an inactive target gene (e.g., see Thomas and Capecchi, 1987 and Thompson, 1989, supra). However, this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body (See generally, Helene, 1991, Anticancer Drug Des., 6(6):569-584; Helene, et al., 1992, Ann. N.Y. Acad. Sci., 660:27-36; and Maher, 1992, Bioassays 14(12):807-815).

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The anti-sense RNA and DNA molecules, ribozymes and triple helix molecules of the invention may be prepared by any method known in the art for the synthesis of nucleic acid molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the RNA molecule. Such DNA sequences may be incorporated into a wide variety of vectors which contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′O-methyl rather than phospho-diesterase linkages within the oligodeoxyribonucleotide backbone.

Methods for introducing polynucleotides into such cells or tissues include methods for in vitro introduction of polynucleotides such as the insertion of naked polynucleotide, i.e., by injection into tissue, the introduction of a SGT4 polynucleotide in a cell ex vivo, the use of a vector such as a virus, (retrovirus, adenovirus, adeno-associated virus, etc.), phage or plasmid, etc. or techniques such as electroporation or calcium phosphate precipitation.

5.12. Uses of SGT4 Protein

The SGT4 gene is expressed in variety of cell and tissue types, particularly neuronal tissues, brain, heart, liver, pancreas and adrenal gland. The SGT4 protein can regulate cellular function by regulating signal transduction pathways, particularly by interacting with and/or modulating the activity of GTP-binding proteins, e.g., GTPases, that are involved in the regulation of signal transduction pathways. The improper regulation of signal transduction mechanisms can result in cancer.

SGT4, truncated SGT4, SGT4 fragments, SGT4 fusion proteins, or antibodies to SGT4 can be used as therapeutics, particularly in the treatment of diseases or conditions involving the aberrant operation of a signal transduction pathway, e.g., cancer. In a preferred embodiment, SGT4 can be used to treat diseases involving aberrant signal transduction in cells and tissues where SGT4 is normally expressed, e.g., brain, heart, liver, pancreas and adrenal gland. Expression or activities of SGT4 may be upregulated or down-regulated depending on the desired outcome.

SGT4 protein inhibitors or anti-SGT4 antibodies may function to directly interfere with SGT4 enzymatic activities, with the binding of SGT4 to its conjugate ligand, or with the interaction of SGT4 with other proteins or molecules involved in signal transduction. Such inhibitors and antibodies can be used in the treatment of various disorders, particularly disorders involving the aberrant regulation or functioning of signal transduction mechanisms, e.g., cancer.

5.13. Formulation and Route of Administration

A SGT4 polypeptide, a fragment thereof or an anti-SGT4 antibody may be administered to a subject per se or in the form of a pharmaceutical or therapeutic composition. Pharmaceutical compositions comprising the proteins of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the protein or active peptides into preparations which can be used pharmaceutically. Proper formulation is dependent upon the route of administration chosen.

For topical administration the proteins of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the proteins of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents. Alternatively, the proteins may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, a composition can be readily formulated by combining the proteins with pharmaceutically acceptable carriers well known in the art. Such carriers enable the proteins to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the proteins may take the form of tablets, lozenges, etc. formulated in conventional manner.

For administration by inhalation, the proteins for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The proteins may also be formulated in rectal or vaginal compositions such as suppositories or retention enemas, e.g, containing conventional suppository bases such as cocoa butter or other glycerides.

In addition to the formulations described previously, the proteins may also be formulated as a depot preparation. Such long acting formulations may be administered by implantation (for example subcutaneously or intramuscularly) or by intramuscular injection. Thus, for example, the proteins may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver the proteins or peptides of the invention. Certain organic solvents such as dimethylsulfoxide also may be employed, although usually at the cost of greater toxicity. Additionally, the proteins may be delivered using a sustained-release system, such as semipermeable matrices of solid polymers containing the therapeutic agent. Various sustained-release materials have been established and are well known by those skilled in the art. Sustained-release capsules may, depending on their chemical nature, release the proteins for a few weeks up to over 100 days. Depending on the chemical nature and the biological stability of the therapeutic reagent, additional strategies for protein stabilization may be employed.

As the proteins of the invention may contain charged side chains or termini, they may be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

5.14. Effective Dosages

SGT4 polypeptides, SGT4 fragments and anti-SGT4 antibodies will generally be used in an amount effective to achieve the intended purpose. The proteins of the invention, or pharmaceutical compositions thereof, are administered or applied in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Dosage amount and interval may be adjusted individually to provide plasma levels of the proteins which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the proteins may not be related to plasma concentration. One having skill in the art will be able to optimize therapeutically effective local dosages without undue experimentation:

The amount of SGT4 administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs. In the case of hypercholesterolemia, other conventional drugs may be used in combination with SGT4 or fragments thereof.

Specific dosages may also be utilized for antibodies. Typically, the preferred dosage is 0.1 mg/kg to 100 mg/kg of body weight (generally 10 mg/kg to 20 mg/kg), and if the antibody is to act in the brain, a dosage of 50 mg/kg to 100 mg/kg is usually appropriate. If the antibody is partially human or fully human, it generally will have a longer half-life within the human body than other antibodies. Accordingly, lower dosages of partially human and fully human antibodies is often possible. Additional modifications may be used to further stabilize antibodies. For example, lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

A therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

Moreover, treatment of a subject with a therapeutically effective amount of a protein, polypeptide or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5 or 6 weeks.

The present invention further encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors known to those or ordinary skill in the art, e.g., a physician. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.

Useful pharmaceutical dosage forms, for administration of the compounds of this invention can be illustrated as follows:

Capsules: Capsules are prepared by filling standard two-piece hard gelatin capsulates each with the desired amount of powdered active ingredient, 175 milligrams of lactose, 24 milligrams of talc and 6 milligrams magnesium stearate.

Soft Gelatin Capsules: A mixture of active ingredient in soybean oil is prepared and injected by means of a positive displacement pump into gelatin to form soft gelatin capsules containing the desired amount of the active ingredient. The capsules are then washed and dried.

Tablets: Tablets are prepared by conventional procedures so that the dosage unit is the desired amount of active ingredient. 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings may be applied to increase palatability or to delay absorption.

Injectable: A parenteral composition suitable for administration by injection is prepared by stirring 1.5% by weight of active ingredients in 10% by volume propylene glycol and water. The solution is made isotonic with sodium chloride and sterilized.

Suspension: An aqueous suspension is prepared for oral administration so that each 5 millimeters contain 100 milligrams of finely divided active ingredient, 200 milligrams of sodium carboxymethyl cellulose, 5 milligrams of sodium benzoate, 1.0 grams of sorbitol solution U.S.P. and 0.025 millimeters of vanillin.

5.15. Toxicity

Preferably, a therapeutically effective dose of the proteins described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p.1).

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims. All references, patents, and patent applications cited herein are hereby incorporated by referenced in their entirety.

1.-20. (cancelled)

21. An isolated nucleic acid molecule comprising the nucleotide sequence described in SEQ ID NOS: 1 or 3.

22. An isolated nucleic acid molecule comprising a nucleotide sequence encoding the amino acid sequence disclosed in SEQ ID NOS: 2 or 4.

23. A recombinant vector comprising a nucleic acid sequence of claim 24.

24. A host cell comprising the recombinant vector of claim 23.


SEQUENCE LISTING (4 sequences)

SEQ ID NO: 1 - DNA, 1116 bp, Homo sapien
  CDS (1)...(1113)
atgggacataaagtggttgtcttcgacatttctgtcatcagagccttgtgggaaactcgtgtcaagaagcacaaagcttggcagaagaaggaggtggaaaggcttgagaagagcgccttggagaagataaaggaggagtggaactttgtggccgaatgcaggaggaagggcatcccccaggctgtatactgcaagaatggcttcatagacaccagcgtgcggcttctggacaagattgaaaggaacactctcacaaggcagagttcacttcccaaggacagaggcaaacggagcagtgcgtttgtgtttgaactttctggggagcactggacggagctcccagattcattgaaggagcagacacacctgagagaatggtacataagcaataccttgattcaaatcattcctacatatattcagttatttcaagcgatgagaattctggatctgccaaaaaaccaaatctcacatcttccagcagaaatcggttgtttgaagaacctgaaagaactcaatgtgggtttcaactatctgaagagcattcctccagaattgggagattgtgaaaatctagagagactggattgttctggaaatctagaattaatggagctgccctttgaattaagtaatttgaagcaagttacatttgtagatatctcagcaaacaagttttccagtgtcccaatctgtgtcctgcggatgtcgaatttgcagtggttggatatcagcagcaataacctgaccgacctgccgcaagatatagacaggctagaggagctgcagagctttctcttgtataaaaacaagttgacctaccttccctattccatgctgaacctgaagaagctcactctgttagtcgtcagtggggaccatttggtggagctcccaactgccctttgtgactcatccacacctttaaaatttgtaagccttatggacaatcctattgataatgcccaatgtgaagatggcaatgaaataatggaaagtgaacgggatcgccaacattttgataaagaagttatgaaagcctatattgaagaccttaaagaaagagaatctgttcccagctataccaccaaagtgtcttttagccttcaactttga

SEQ ID NO: 2 - protein, 371 aa, Homo sapien
MGHKVVVFDISVIRALWETRVKKHKAWQKKEVERLEKSALEKIKEEWNFVAECRRKGIPQAVYCKNGFIDTSVRLLDKIERNTLTRQSSLPKDRGKRSSAFVFELSGEHWTELPDSLKEQTHLREWYISNTLIQIIPTYIQLFQAMRILDLPKNQISHLPAEIGCLKNLKELNVGFNYLKSIPPELGDCENLERLDCSGNLELMELPFELSNLKQVTFVDISANKFSSVPICVLRMSNLQWLDISSNNLTDLPQDIDRLEELQSFLLYKNKLTYLPYSMLNLKKLTLLVVSGDHLVELPTALCDSSTPLKFVSLMDNPIDNAQCEDGNEIMESERDRQHFDKEVMKAYIEDLKERESVPSYTTKVSFSLQL

SEQ ID NO: 3 - DNA, 681 bp, Homo sapien
  CDS (1)...(678)
atgagaattctggatctgccaaaaaaccaaatctcacatcttccagcagaaatcggttgtttgaagaacctgaaagaactcaatgtgggtttcaactatctgaagagcattcctccagaattgggagattgtgaaaatctagagagactggattgttctggaaatctagaattaatggagctgccctttgaattaagtaatttgaagcaagttacatttgtagatatctcagcaaacaagttttccagtgtcccaatctgtgtcctgcggatgtcgaatttgcagtggttggatatcagcagcaataacctgaccgacctgccgcaagatatagacaggctagaggagctgcagagctttctcttgtataaaaacaagttgacctaccttccctattccatgctgaacctgaagaagctcactctgttagtcgtcagtggggaccatttggtggagctcccaactgccctttgtgactcatccacacctttaaaatttgtaagccttatggacaatcctattgataatgcccaatgtgaagatggcaatgaaataatggaaagtgaacgggatcgccaacattttgataaagaagttatgaaagcctatattgaagaccttaaagaaagagaatctgttcccagctataccaccaaagtgtcttttagccttcaactttga

SEQ ID NO: 4 - protein, 226 aa, Homo sapien
MRILDLPKNQISHLPAEIGCLKNLKELNVGFNYLKSIPPELGDCENLERLDCSGNLELMELPFELSNLKQVTFVDISANKFSSVPICVLRMSNLQWLDISSNNLTDLPQDIDRLEELQSFLLYKNKLTYLPYSMLNLKKLTLLVVSGDHLVELPTALCDSSTPLKFVSLMDNPIDNAQCEDGNEIMESERDRQHFDKEVMKAYIEDLKERESVPSYTTKVSFSLQL